(19) United States
(12) Patent Application Publication
(10) Pub. No.: US 2022/0008193 A1
(43) Pub. Date: Jan. 13, 2022

(54) FASTENING DEVICE FOR AN INTRAOCULAR LENS

(71) Applicant: MEDIZINISCHE UNIVERSITÄT WIEN, Wien (AT)

(72) Inventor: Stefan SACU, Wien (AT)

(21) Appl. No.: 17/413,359

(22) PCT Filed: Dec. 27, 2018
(86) PCT No.: PCT/AT2018/000101

§ 371 Date: Jun. 11, 2021

(51) Int. Cl. A61F 2/16

(52) U.S. Cl. CPC A61F 2/16; A61F 2220/0091; A61F 2220/0016; A61F 2002/1683

(57) ABSTRACT

A fastening device for fixing an intraocular lens having protruding support elements in the eye of a patient comprises at least one tip suitable for penetrating the human ciliary body and preferably suitable for penetrating the human sclera and is connected or connectable to a protruding support element.

The present invention relates to a fastening device for fixing an intraocular lens having protruding support elements in the eye of a patient.

The invention further relates to a kit of parts comprising an intraocular lens having protruding support elements and at least one fastening device which can be connected to one of the protruding support elements.

Cataracts are one of the most common diseases of the human eye in old age. By implanting an artificial lens instead of the cloudy natural lens, around 90% of patients can restore 50% to 100% of their visual performance. Cataract surgery is therefore the most common surgery on the human eye. In Germany alone, around 600,000 of this type of surgery are carried out every year.

In cataract surgery, the cloudy natural lens is removed and an artificial intraocular lens (IOL) with suitable refractive power is inserted. While in the past the entire natural lens was removed, it is now common practice to leave the posterior and lateral lens capsules in the eye.

In addition to the lens part providing the refractive power, the artificial intraocular lens usually has protruding support elements, so-called “haptics”, which are intended to be supported in the area of the ciliary sulcus in order to keep the IOL centered in the area of the lens capsule. Although this works well in most cases, in cases with capsular bag injuries or in cases with a weak suspension apparatus of the lens capsule it can happen that the IOL cannot be implanted in the intended place or does not remain in the lens capsule bag and slips and/or tilts, which is of course to be regarded as unacceptable.

In cases with capsular bag defects during cataract surgery, ophthalmic surgeons currently use two treatment options. With the method called “Iris fixation of IOL implant”, however, relatively large incisions have to be made in the cornea. The risks of this method are damage to the cornea and the iris and, in the long term, the formation of glaucoma. Impaired pupillary response is another disadvantage of this method. For this reason, the “sulcus-trans-scleral IOL suture fixation” method is increasingly being practiced in order to be able to reproduce the desired visual acuity in aphakic patients or patients whose eyes have insufficient capsule support. With this surgical technique, the sclera has to be partially unfolded and then sutured again in order to sew the haptics of IOLs to parts of the sclera. This technique is surgically demanding and carries a certain risk of introcular bleeding. In addition, this procedure can also result in tilting, decentering and/or slipping of the IOL into the vitreous space. A major disadvantage of this method is that IOLs with thick haptics cannot be implanted because of the unacceptably high decentration rate, although IOLs with thick haptics are increasingly preferred by ophthalmic surgeons because of the possibility of simpler injection techniques.

Yokrat Ton et al. published in 2007 an anchoring device for an intraocular lens that anchors in the ciliary sulcus. This device and the associated type of fixation were identified as effective in the animal model.

The present invention aims to create a fastening device for fixing a intraocular lens that comprises protruding support elements in the eye of a patient, which allows a better and more reliable fixing of an intraocular lens in the human eye and which in particular allows a tilted and/or slipped intraocular lens to be permanently repositioned, e.g. in a revision surgery, or to implant an artificial intraocular lens primarily safely against slipping and/or tilting.

To achieve this object, the fastening device according to the invention comprises at least one tip which is suitable for penetrating the human ciliary body and preferably also for penetrating the human sclera and which is or can be connected to a protruding support element.

Because a fastening device is connected to the haptics, which is provided with a tip for penetrating anatomical structures of the eye in the area of the lens or the lens capsule, the artificial intraocular lens cannot only be fixed by the haptics, which serve as pure support elements, which are elastically supported on the sulcus. Rather, the haptics and thus the intraocular lens can be pinned to anatomical structures of the eye using a type of needle, so that subsequent slipping or tilting of the intraocular lens can practically be ruled out. Other important advantages of this method are the possibility of using all types of open-loop haptics (regardless of the material and thickness of the haptics) and of course the better centering of the IOL, which is an important problem with the “Sulcus-trans-scleral IOL suture fixation” method.

In connection with the present invention, penetration means that the tip can penetrate from the inside through the ciliary body or the sclera. The tip does not have the strength to pierce said anatomical structures itself. Rather, it is provided that said structures are pierced or perforated with special needles or lances in order to grip the tip with a special tool from the outside and pull it out through the pierced opening. The fastening device according to the invention is preferably designed in one piece with the protruding support elements, i.e. the haptics, that is to say that the artificial intraocular lens is already produced with the fastening device according to the invention on the protruding support elements.

The fastening device according to the invention is primarily intended as a medical product for ophthalmological revision surgery, but can also be used in the primary care of cataract patients and can be attached to the haptics of intraocular lenses to supplement them before being used. For this purpose, the fastening device according to the invention has a connecting structure for connecting the fastening device to a protruding support element of the intraocular lens, as corresponds to a preferred embodiment of the present invention. The connecting structure is preferably designed as a hinge-like openable clamping region into which a protruding support element can be inserted and clamped. The tip is then passed through the ciliary sulcus or the sclera and appropriately anchored.

According to a preferred embodiment of the present invention, the connecting structure is made of a different material than the at least one tip. In this way, the two essential regions of the fastening device according to the invention—the tip and the connecting structure—can be produced from materials that are adapted in each case in order to best serve their respective purpose.

For better anchoring of the tip in the ciliary sulcus or in the sclera, the fastening device according to the invention can preferably be developed in such a way that the at least one tip has at least one barbed hook. In this way the tip is self-locking and allows the surgery to be performed quickly.

According to a preferred embodiment of the present invention, the fastening device has two tips which are connected to one another via a common cavity extending within in the two tips. With this particular and preferred embodiment of the present invention, the fastening device according to the invention can be sewn to the eye if both tips are passed through the mentioned anatomical structures and a thread is drawn through the common cavity in the two tips, which is then suitably knotted.

In order to be able to grasp and manipulate the fastening device according to the invention reliably and without damaging it during surgery, the fastening device has, adjacent to the connecting structure, at the end of the fastening device opposite the tip, a holding region for engagement by surgical tweezers, as in correspondence to a preferred embodiment of the present invention. The holding region is preferably made of a different material than the connecting structure and/or the tip and can therefore be designed with regard to the material according to the requirements of stability and toughness for multiple gripping during surgery.

The surgical tool according to the invention for manipulating the fastening device according to the invention comprises a shaft and a coupling for the tip so that the surgeon can insert the surgical tool into the eye through the perforation that was previously created in the sclera, grasp the tip of the fastening device intraocularly and pull it with the shaft out through the perforation.

The invention is explained in more detail below with reference to an exemplary embodiment shown in the drawing. In the drawings

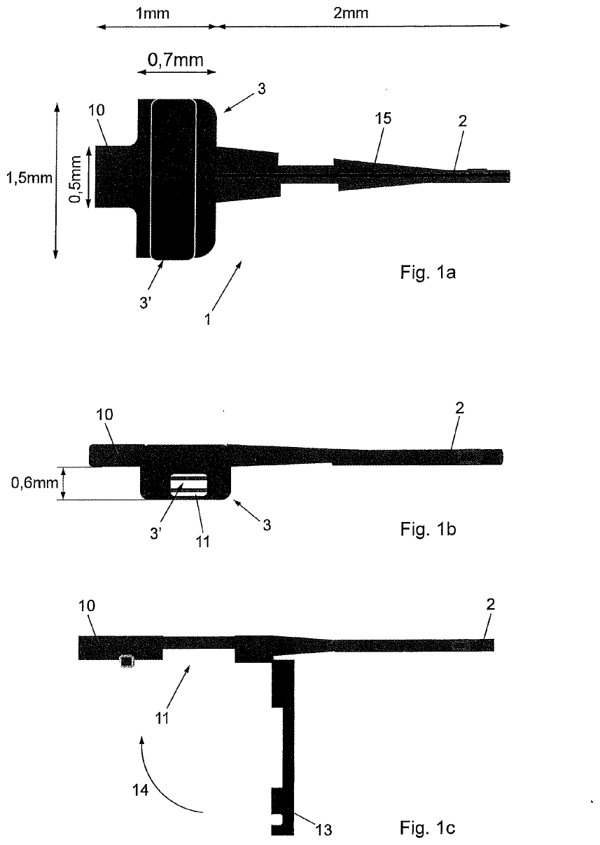

FIG. 1a-1c show schematic representations of a fastening device according to the invention,

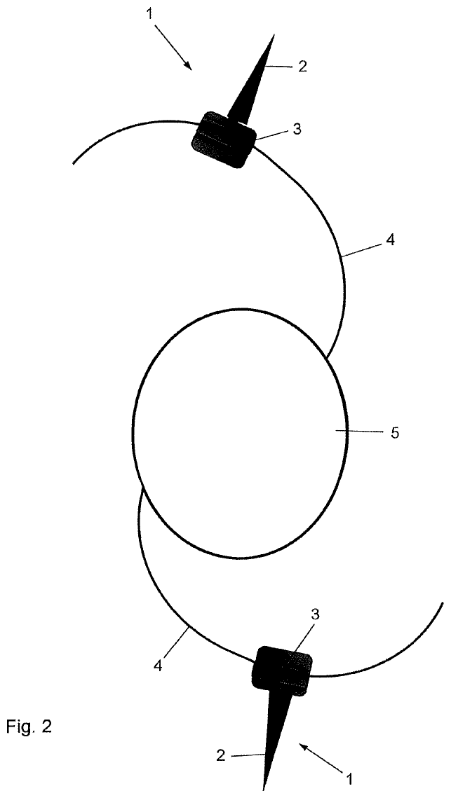

FIG. 2 shows a schematic representation of the interaction of a fastening device according to the invention with an intraocular lens having protruding support elements,

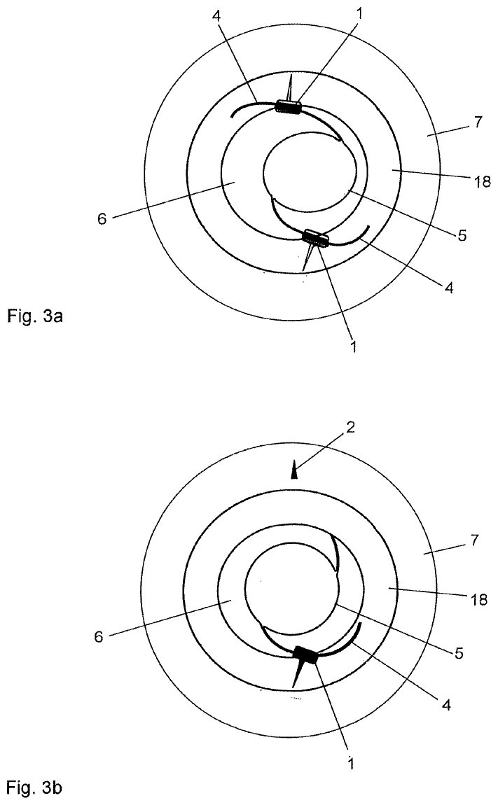

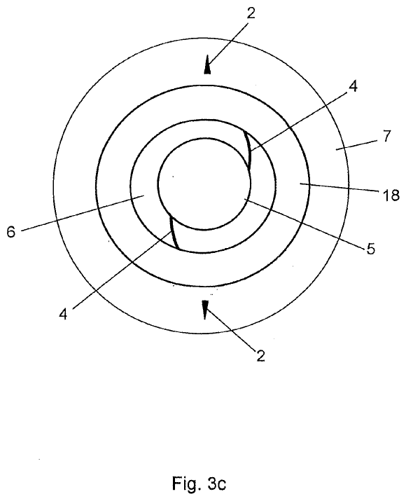

FIG. 3a-3c show a schematic representation of the use of a fastening device according to the invention on the eye,

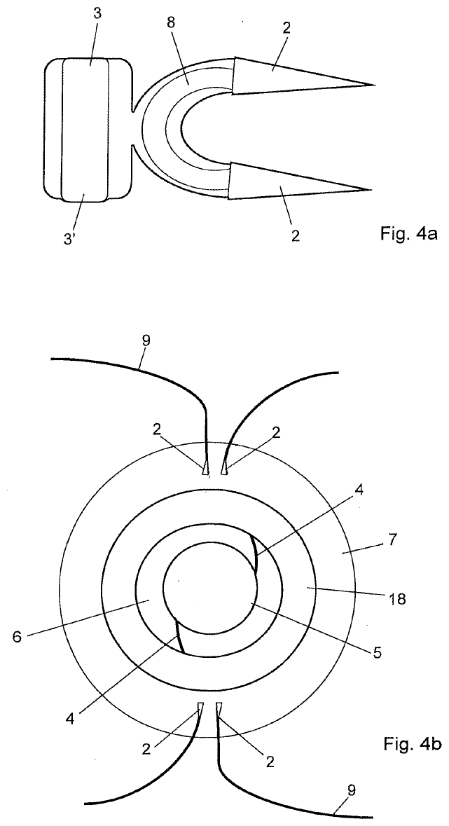

FIG. 4a shows a schematic representation of a preferred variant of a fastening device according to the invention,

FIG. 4b shows an illustration of the application of the fastening device according to FIG. 4 on the eye,

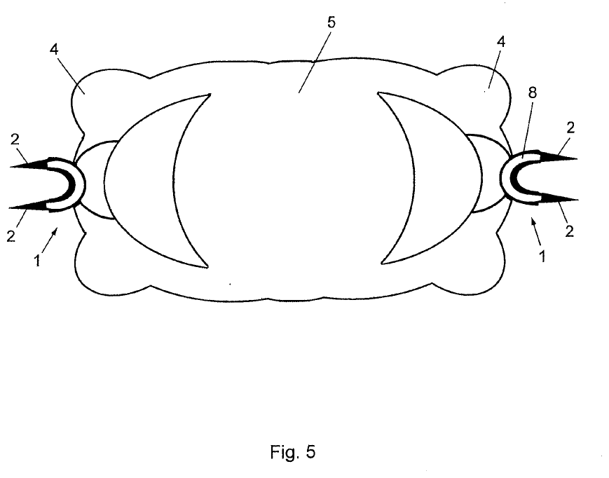

FIG. 5 shows an illustration of a lens configuration with a fastening device according to the present invention and

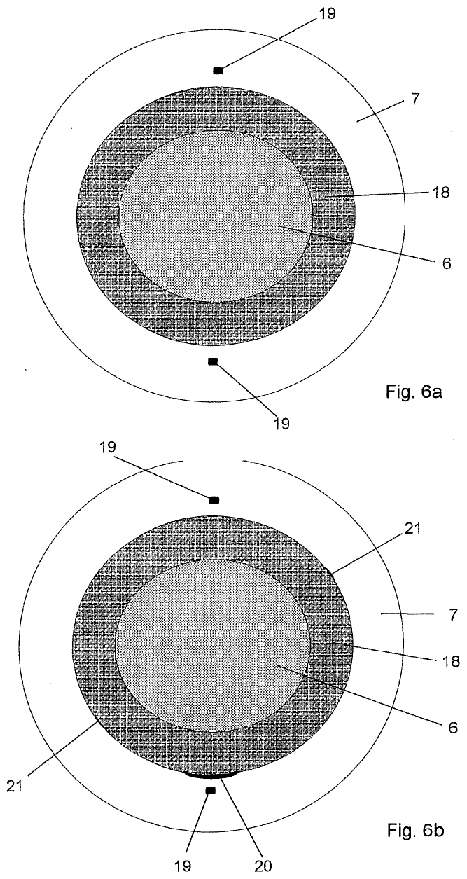

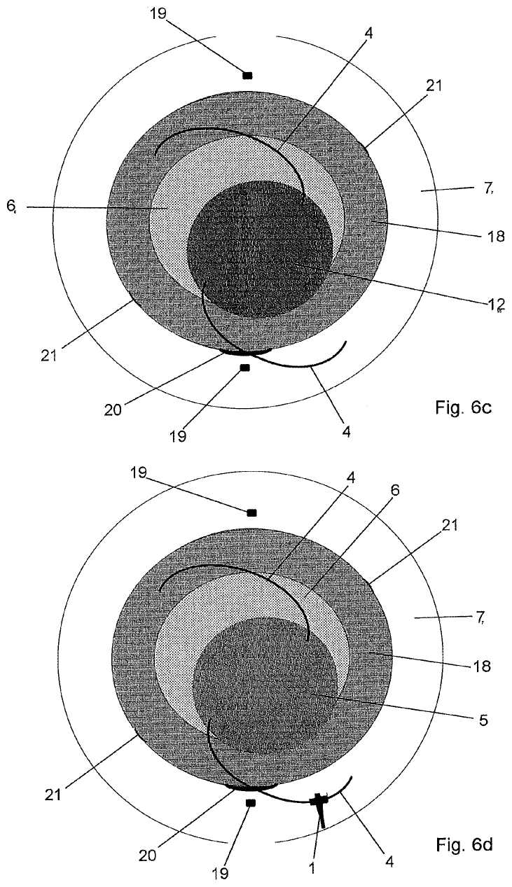

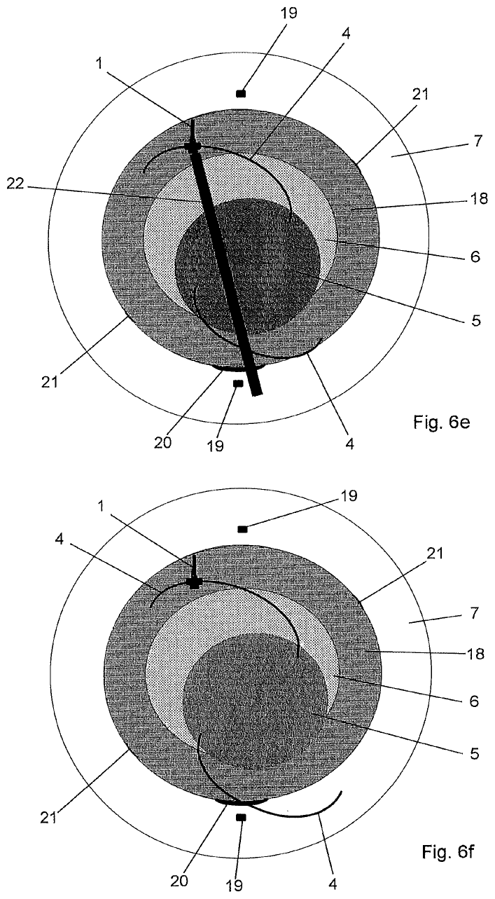

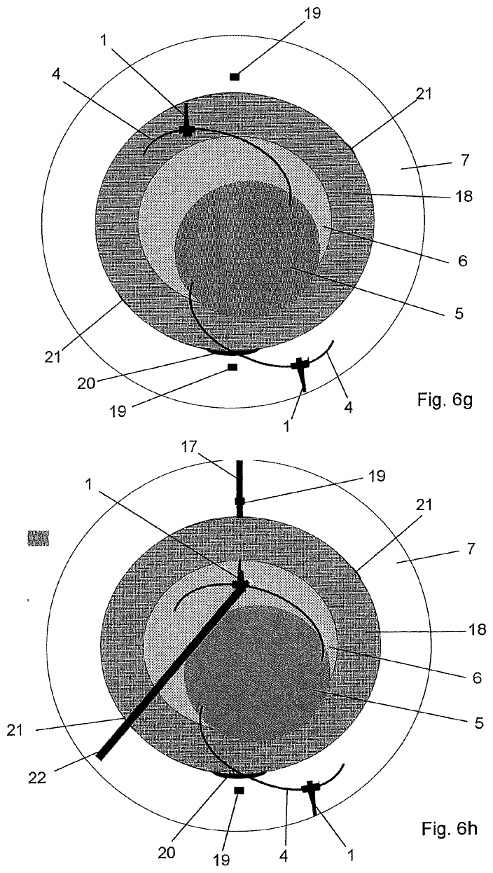

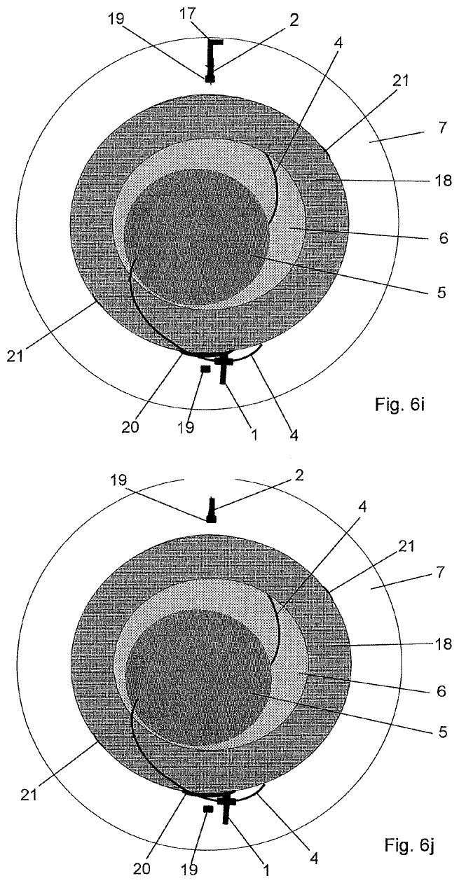

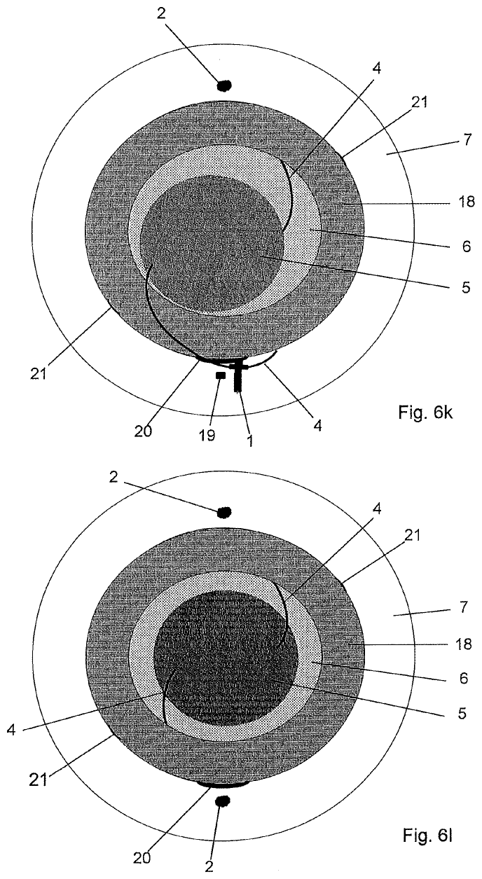

FIGS. 6a to 6l show an illustration of an operation sequence using the surgical tool according to the invention together with the fastening device according to the invention.

In FIG. 1a, a fastening device according to the invention is generally designated by the reference number 1. The fastening device 1 has a tip 2 which is suitable for penetrating the human ciliary body or the human sclera. In connection with the present invention, penetration means that the tip 2 can penetrate through the ciliary body or the sclera. The fastening device 1 has a connecting structure 3 with which the fastening device 1 can be connected with so-called haptics, i.e. with the protruding support elements of an artificial intraocular lens. For this purpose, the connecting structure 3 according to a preferred embodiment has a clamping region 3′. The fastening device 1 according to the invention also has a holding region 10 at which the fastening device 1 can be gripped, for example, by tweezers, and manipulated accordingly during surgery. Reference numeral 15 denotes an axis marking which is intended to help in particular to position toric IOLs appropriately and to determine a tilting of the IOL due to any incorrect manipulation during the surgical treatment and to correct it immediately.

In the side view of the fastening device 1 according to the invention according to FIG. 1b, it can be seen that the connecting structure 3 has a passage 11 for a protruding support structure 4, which forms the clamping region 3′. In FIG. 1c it can be seen that the clamping region 3′ can be opened by a clamping cover 13, so that the protruding support structure can be inserted into the passage 11 and fixed in the direction of the arrow 14 by closing the clamping cover 13.

In the following figures, the same or equivalent parts are provided with the same reference numerals. FIG. 2 shows that two pieces of the fastening device 1 according to the invention are connected to the haptics 4, i.e. to the protruding support elements 4 of an artificial intraocular lens 5, the protruding support elements 4 being clamped in the fastening region 3.

In FIG. 3a it is shown how the artificial intraocular lens 5 with protruding support elements 4, on which the fastening device 1 according to the invention is fixed, is inserted or placed in front of the pupil 6 and in front of the iris 18. FIG. 3b shows a state in which the tip 2 of the fastening device 1 attached to one of the two protruding support elements 4 passes through the sclera 7. FIG. 3c shows the state after the artificial intraocular lens 5 has been fixed behind the pupil 6, the tip 2 of the fastening device 1 attached to the other of the two protruding support elements 4 also passing through the sclera 7. It should be understood that the tips 2 of the fastening devices 1 are firmly connected to the eye and that the artificial intraocular lens 5 is securely positioned behind the pupil 6 through the intermediary of the protruding support elements 4.

FIG. 4a shows a variant of the present invention in which the fastening device 1 has two tips 2 which are connected to one another via a common cavity 8 extending within the two tips 2. As can be seen in FIG. 4b, the cavity opening at the tips 2 can be used to pull a surgical thread 9 through the tips 2 passing through the sclera and to fix the fastening device 1 according to the invention in such a way as to secure the artificial intraocular lens 5.

In FIG. 5 it can be seen that the fastening device 1 according to the invention can be formed in one piece with the protruding support elements 4, i.e. the haptics 4, that is, the artificial intraocular lens 5 is already manufactured with the fastening device 1 according to the invention on the protruding support elements 4.

The individual steps of the surgical method made possible by the invention are shown in FIGS. 6a to 6l.

In a first step (FIG. 6a), two perforations 19 in a diametrically opposite arrangement, i.e. offset by 180°, are pierced into the sclera 7 at a distance of approximately two millimeters from the limbus (transition from the cornea to the sclera 7). Then (FIG. 6b) a first incision 20 is made in the cornea in the region of the transition to the sclera 7, via which the IOL can subsequently be introduced in front of the pupil 6 and the iris 18. The first incision 20 is approximately 3 millimeters long. Two second incisions with a size of approximately 0.6 millimeters are designated by 21, through which a fine manipulation instrument can later be introduced. Thereafter, the intraocular lens 5 with the haptics 4 or the protruding support elements 4 is introduced in front of the iris 18 (FIG. 6c), with a protruding support element 4 still protruding from the incision 20. A first fastening device 1 according to the invention is then attached to the protruding support element 4 (FIG. 6d), the IOL 5 with the haptics 4 rotated with the aid of a fine manipulator 22 introduced through the incision 20 (FIG. 6e), and the second protruding support element 4 is led out of the incision 20 (FIG. 6f) so that a second fastening device 1 according to the invention can be clamped to the protruding support element 4 (FIG. 6g), preferably at the same distance from the free end of the support element 4 as with the other fastening device. Then the manipulation instrument 22 is on the one hand introduced via the second incision 21 behind the pupil 6 and on the other hand an external surgical tool 17 is led through the scleral perforation 19 and the fastening device 1 according to the invention is connected to the external surgical tool 17 in the eye. The connection between the tip 2 and the surgical tool 17 is made by a coupling (not shown). The tip 2 of the fastening device 1 according to the invention is guided to the outside through the perforation 19 and then uncoupled from the external tool 17 (FIGS. 6h and 6i, 6j) and the same is carried out with the second fastening device 1. The two tips 2 guided through the sclera 7 are deformed by cautery and thereby fixed in a form-fitting manner. (FIGS. 6k and 6l).

1. A fastening device for fixing an intraocular lens having protruding support elements in an eye of a patient, which fastening device comprises at least one tip suitable for penetrating the human ciliary body and suitable for penetrating the human sclera and is connected or connectable to one of said protruding support elements.

2. The fastening device according to claim 1, characterized in that the fastening device has a connecting structure for connecting said at least one tip to one of said protruding support elements of the intraocular lens, the connecting structure being configured as a hinge-like openable clamping region.

3. The fastening device according to claim 2, characterized in that the connecting structure is made from a different material than the at least one tip.

4. The fastening device according to claim 1, characterized in that the at least one tip comprises at least one barbed hook.

5. The fastening device according to claim 1, characterized in that the fastening device has two tips which are connected to one another via a common cavity extending within the two tips.

6. The fastening device according to claim 2, characterized in that the fastening device comprises, adjacent to the connecting structure, at an end of the fastening device opposite the at least one tip, a holding region for engagement by surgical tweezers.

7. The fastening device according to claim 6, characterized in that the holding region is made of a different material than the connecting structure and/or the at least one tip.

8. A kit of parts comprising the intraocular lens having protruding support elements and at least one fastening device according to claim 1, which can be connected to one of the protruding support elements.

9. A surgical tool for manipulating the fastening device according to claim 1, comprising a shaft and a coupling for the at least one tip.

10. The fastening device according to claim 2, characterized in that the at least one tip comprises at least one barbed hook.

11. The fastening device according to claim 2, characterized in that the fastening device has two tips which are connected to one another via a common cavity extending within the two tips.

12. The fastening device according to claim 5, wherein the two tips are connected to a connecting structure for connecting said two tips, via the common cavity, to one of said protruding support elements.
